(19) United States
(12) Patent Application Publication
(10) Pub. No.: US 2003/0170285 A1
(43) Pub. Date: Sep. 11, 2003

(54) Delivery of tissue engineering media

(76) Inventors: William S. Veazey, Gainesville, FL (US); Karen Moore, Gainesville, FL (US)

Correspondence Address: Miles & Stockbridge, Suite 500, 1751 Pinnacle Drive, McLean, VA, 22102-3833, US

(21) Appl. No.: 10/292,797

(22) Filed: Nov. 13, 2002

Related U.S. Application Data
(60) Provisional application No. 60/331,261, filed on Nov. 13, 2001.

(51) Int. Cl. A61K 45/00; A61F 2/00

(52) U.S. Cl. 424/423; 424/93.7

(57) ABSTRACT

The delivery of tissue engineering media using an airbrush.

William S. Veazey, D M D

Nov. 7, 2002

Average Delivery Values for Cell Delivery Study using Tissue
Engineering Media Delivery System

| | Fine Nozzle | Medium Nozzle | Large Nozzle |
|---|---|---|---|
| | 312 microns | 494 microns | 746 microns |
| 6 PSI | 86% | 87% | 94% |
| 8 PSI | 76% | 82% | 93% |
| 10 PSI | 65% | 86% | 89% |
| 14 PSI | 60% | 66% | 69% |
| 18 PSI | 37% | 62% | 56% |

Procedure: Bovine dermal fibroblasts were delivered into a 6-well tissue culture plate using a Badger 100 G airbrush having fine, medium and large nozzles at air pressures 6, 8, 10, 14 and 18 psi. Cells were delivered in a concentration of 200,000 cells per 1 mL of media; 1 mL of this cell suspension was delivered for each repetition. The cells were washed in Hanks Balanced Salt Solution (HBSS) or Phosphate Buffered Saline (PBS). Trypsin (0.25%) was used to detach the cells from the cell culture plate before delivery. Cells were re-suspended in a 50%-50% Dulbecco's Modified Eagle's Medium (DMEM) and F-12 media (one actually purchases the media in this concentration, called DMEM/F12) with 10% Fetal Bovine Serum (FBS), 2 mM L-Glutamine and 0.1 mM 2-Me.

It should be stated that cells could be delivered in a variety of media, and one skilled in the art recognizes that one could use HBSS or PBS alone, or could use DMEM/F12 alone, or any other type of vital media to deliver cells.

Cell vitality was measured using a trypan blue exclusion assay.

Thickness of deposition was not measured. The 1 mL of airbrush delivered media spread in the tissue culture well as media would normally do.

### BACKGROUND OF THE INVENTION

1. Field of the Invention

The invention relates to methods and systems for the delivery of tissue engineering media to sites in or within the body of human or non-human animals.

2. Description of the Prior Art

The term, “tissue engineering” as used herein, comprises the repair, replacement, or regeneration of damaged or diseased tissues by the management of cells, the construction of artificial implants, or the manufacture of substitutes. An example is the process known as “tissue induction,” whereby 2½- and 3-dimensional polymer or mineral scaffolds without cells are implanted in a patient. In tissue induction, tissue generation occurs by the ingrowth of surrounding tissue into the structure.

In another method known as “cell transplantation”, scaffolds are seeded with cells, cytokines, and other growth-related molecules, followed by culturing. The seeded scaffolds are then implanted to induce the growth of new tissue. Cultured cells are infused into a biodegradable or non-biodegradable scaffold, which is implanted directly in the patient, or first cultured in a reactor wherein the cells proliferate before implantation. The cell-scaffold construct may be implanted directly in the patient, thus using the patient's body as an in-vivo bioreactor. Once implanted, in-vivo cellular proliferation and, in the case of absorbable scaffolds, concomitant bio-absorption of the scaffold, proceeds.

There exist numerous techniques for manufacturing scaffolds for tissue generation depending upon the type of tissue ultimately desired. In one procedure hydroxyapatite is machined to a desired shape. Another technique, known as “fiber bonding”, involves preparing a mold in the shape of the desired scaffold and placing fibers, such as polyglycolic acid (PGA) into the mold and embedding the PGA fibers in, e.g., a poly (L-lactic acid) (PLLA)/methylene chloride solution. The solvent is evaporated, and the PLLA-PGA composite is heated above the melting temperatures of both polymers. The PLLA is then removed by selective dissolution after cooling, leaving the PGA fibers physically joined at their intersections.

Presently employed systems for the delivery of tissue constructs also include 3-D dot matrix “tissue printers”, aerosolized collagen spray systems (U.S. Pat. No. 5,645,820), engineered tissue sheets (U.S. Pat. Nos. 6,146,892; 6,143,293 and 6,103,255), engineered cartilage grafts (U.S. Pat. Nos. 6,287,340; 5,962,325 and 5,902,741), periodontal guided tissue regeneration barrier membranes (U.S. Pat. No. 4,961,707), and the like.

U.S. Pat. No. 5,686,091 discloses a method in which biodegradable porous polymer scaffolds are prepared by molding a solvent solution of the polymer under conditions permitting spinodal decomposition, followed by quenching of the polymer solution in the mold and sublimation of the solvent from the solution.

U.S. Pat. No. 5,723,508 discloses a method in which biodegradable porous polymer scaffolds are prepared by forming an emulsion of the polymer, a first solvent in which the polymer is soluble, and a second polymer that is immiscible with the first solvent, and then freeze-drying the emulsion under conditions that do not break the emulsion or throw the polymer out of solution.

Solvent casting is one of the most widely used processes for fabricating scaffolds of degradable polymers (see Mikos et al., Polymer, 35, 1068-77, (1994); de Groot et al., Colloid Polym. Sci., 268, 1073-81 (1991); Laurencin et al., J Biomed. Mater. Res., 30, 133-8 (1996)). U.S. Pat. No. 5,514,378 discloses the basic procedure in which a polymer solution is poured over a bed of salt crystals. The salt crystals are subsequently dissolved away by water in a leaching process. De Groot et al. disclose a modified leaching technique in which the addition of a co-solvent induces a phase separation of the system upon cooling through liquid-liquid demixing.

The method known as “melt molding”, comprises placing a mixture of fine PLGA powder and gelatin microspheres in a mold and heating the system to the glass-transition temperature of the polymer. The PLGA-gelatin composite is removed from the mold and gelatin microspheres are leached out by selective dissolution. Other scaffold manufacturing techniques include polymer/ceramic fiber composite foam processing, phase separation, and high-pressure processing.

Tissue engineers face the problem of incrementally building up the scaffold and implanting the cells and growth factors in the scaffold. One process known as solid freeform fabrication entails computer-aided-design and computer-aided-manufacturing (CAD/CAM) methodologies which have been used in industrial applications to quickly and automatically fabricate arbitrarily complex shapes. Such processes construct shapes by incremental material buildup and fusion of cross-sectional layers.

A three-dimensional printing process is another method for creating scaffolds for engineered tissue. An ink-jet printing mechanism scans a powder surface and selectively injects a binder therein, which joins the powder together, into those areas defined by the geometry of the cross-section. That layer is lowered and the next layer of powder is applied by the ink jet. This of 3D printing process has been used for fabricating biomaterial structures out of bovine bone and biopolymers.

“Membrane lamination” is another technique used for constructing three-dimensional biodegradable polymeric scaffolds. A contour plot of the three-dimensional shape is prepared and porous PLLA or PLGA membranes having the shapes of the contour are then manufactured using the solvent-casting and particulate-leaching technique. Adjacent membranes are bonded together by coating chloroform on their contacting surfaces. The final scaffold is thus formed by laminating the constituent membranes in the proper order to create the desired three-dimensional shape.

Some cell culture and transplantation techniques incorporate cells directly in collagen matrices before the collagen is molded into the final scaffold shape. Further, 3D printing techniques can create nonhomogeneous microstructures. One approach suggested for preparing three-dimensional synthetic tissues is described in Klebe, “Cytoscribing: A Method for Micropositioning Cells and the Construction of Two- and Three-Dimensional Synthetic Tissues,” Experimental Cell Research 179 (1988) pp. 362-373. Klebe discusses the use of ink jet printing techniques to selectively deposit cell adhesion proteins on a substrate. This technique uses monolayers of cells growing on thin sheets of collagen. The sheets can be attached to one another by gluing them together with collagen.

Still, while all of the existing scaffold fabrication methods can be useful techniques for specific applications, a general system for creating large scale, heterogeneous three dimensional scaffold systems, capable of supporting 3-dimensional cell culture and vascularization does not exist.

Significant advances have been made in developing materials other than scaffolds and the like for engineering tissue. For example, a modest amount of research has been done in the area of clinical delivery of tissue engineering media. The current clinical standards for delivering tissue engineering media are injection via syringe and trimming pre-fabricated materials to fit the surgical field. Injection of media works well for internal surgical procedures, but injection is ineffective in delivering layers of fluid media to a surface wound, such as an abrasion, ulcer, periodontal defect, or an open surgical field. Prefabricated materials that are trimmed to fit a wound site are not precise by virtue of the “trim to fit” concept. Moreover, wounds do not come in standard sizes. Delivery of tissue engineering media by syringe and using prefabricated scaffolds are inefficient and waste expensive materials. There is a need for a method to deliver fluid tissue engineering media and cells to a wound field in thin layers.

It is an object of the present invention to provide a novel system for delivering tissue engineering media to both in vitro and in vivo sites where needed.

### SUMMARY OF THE INVENTION

One embodiment of the invention relates to a method for tissue engineering comprising the steps of:

(1) providing a flowable biocompatible composition; and

(2) delivering the resultant combination to a substrate via fluid propulsion, the flowable composition comprising a tissue engineering medium and the composition being delivered by the airbrush under conditions that are not substantially deleterious to the tissue engineering medium.

### BRIEF DESCRIPTION OF THE DRAWINGS

FIGS. 1 and 2 are schematic representations of the air-brush tissue engineering media delivery systems of the invention.

FIG. 3 depicts typical airbrush nozzle diameters.

FIG. 4 depicts the effect of air brush delivery parameters on cell viability.

FIG. 5 depicts the effect of air delivery pressure and nozzle diameter on viscosity of air brush delivered alginate hydrogel.

### DETAILED DESCRIPTION OF THE INVENTION

The present invention is predicated on the use of an airbrush to deliver tissue engineering media in vitro or in vivo to a desired substrate. Conventional wisdom would appear to dictate that an airbrush would be too harsh to deliver such sensitive materials without destroying their innate characteristics. However, by varying the conditions of delivery such as, for example, nozzle diameter, the airbrush is capable of delivering cells with delivery survival rates approaching 94%. Hydrogels may also be delivered without a significant change in viscosity, indicating a low shear effect employing airbrushes.

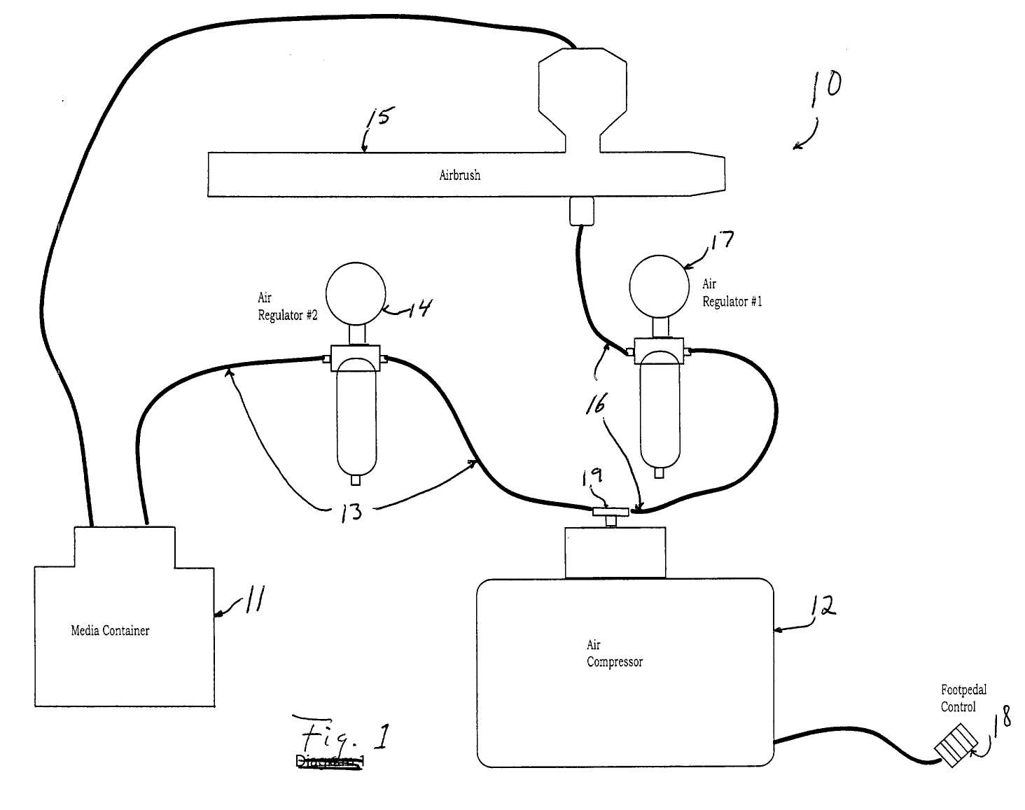

Referring to the drawings, FIG. 1 depicts a typical system 10 according to the invention for delivery by an airbrush of tissue engineering media. Tissue engineering media is loaded into the media container 11 of the system 10. Air compressor 12 is connected to media container 11 via lines 13 and the air pressure therein is regulated by air regulator 14 for delivery of the media to airbrush 15. The compressor 12 is also connected via lines 16 to the air brush 15. Air pressure in lines 16 for regulating the flow of air through airbrush 15 for delivery of the media to the desired substrate is regulated by air regulator 17. The air compressor 12 is most conveniently operated by depressing foot pedal control means 18.

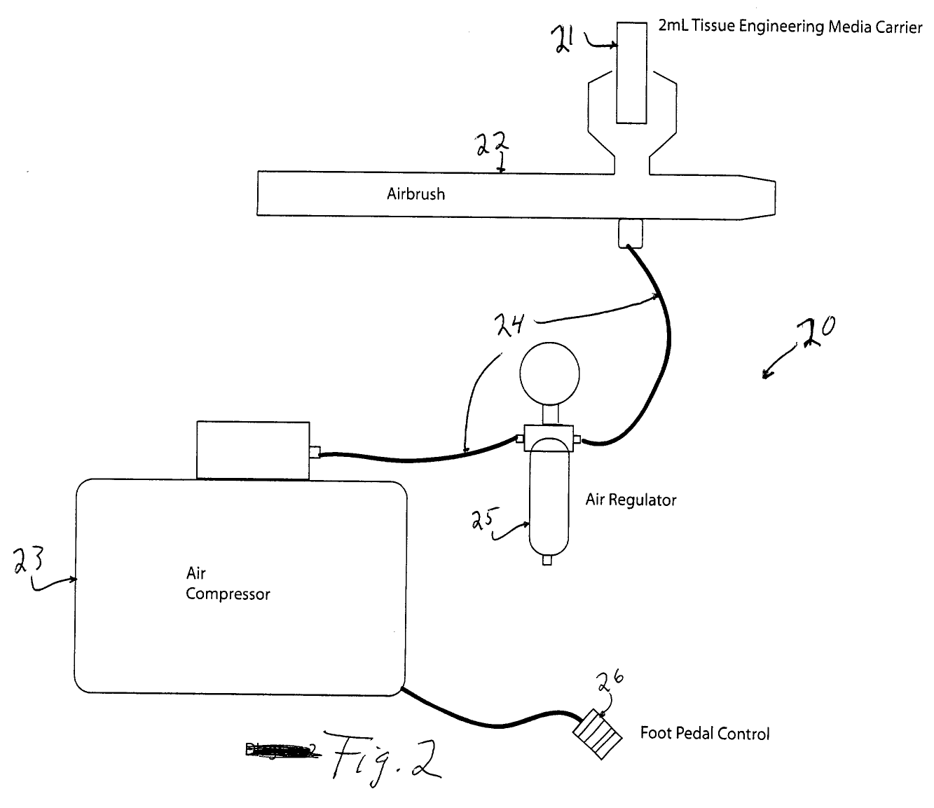

The system of FIG. 1 is most suitable for the delivery of large volumes of media. For smaller volumes of media or where space constraints dictate, the system 20 of FIG. 2 may be more appropriate. Therein, the media container 21 is mounted directly on the airbrush 22 and is designed to slowly meter the media into the airbrush 22 for delivery to the desired substrate. Preferably, the container 21 comprises a microcentrifuge tube for delivery of the tissue engineering media to the airbrush because microcentrifuge tubes fit well into the airbrush media chamber. It will be understood, however, by those skilled in the art that the tube may be of any type such as, for example, a small scintillation tube, a sample collection tube or a tube machined out of a desirable material.

As in the case of the system of FIG. 1, air compressor 23 delivers air under pressure through lines 24, regulated by regulator 25, to airbrush 22. Again, the compressor 23 is operated via foot pedal 26. Air delivered from the compressor 12 to the container 11 and airbrush 15, respectively, is split upon leaving the compressor 11 by T-adaptor 19 to lines 13 and 16.

In carrying out the method of the invention, the desired air pressure is set, usually at a point between 6-18 psi and the foot pedal of the compressor is depressed. The control of the airbrush is operated to deliver the desired amount of media. Media is delivered, e.g., via a sweeping action of the forearm and wrist in the direction the airbrush is pointed. Media is generally delivered at room temperature; however, those skilled in the art will realize that the system is operable at any suitable or convenient temperature that is not deleterious to the operation. Air pressures for delivery are from 6 PSI through 18 PSI. Nozzle sizes are generally 312 and 708 microns (inner nozzle/outer nozzle) for the fine nozzle, 494 microns and 968 microns for the medium nozzle, and 746 microns and 1184 microns for the large nozzle. One or two ml aliquots are usually delivered using the small volume media carrier, and 1 ml to 30 ml of media are generally delivered with the large volume media carrier.

Since not all desired operations require the delivery of the same amounts of media, the invention contemplates two types of system: a low volume media carrier and a larger, variable volume media carrier. The low volume media carrier is typically a 2 ml microcentrifuge tube. A typical adaptor to connect the microcentrifuge tube to the airbrush is such that 12 gauge hypodermic stock pierces the top of a microcentrifuge tube, and media flow into the airbrush is due to air flowing through the airbrush.

When larger amounts of tissue engineering media are needed, a larger media carrier can be connected to the airbrush. One air compressor is connected individually to the media carrier and to the airbrush by using a T-adaptor. To control media delivery into the airbrush separately from airflow into the airbrush, each of these two components is controlled by separate regulators, which are connected to the T-adaptor. Air enters the media carrier and displaces the media, which flows through a connection hose into the airbrush. Thus, the air regulator controls airflow to the media carrier, which in turn regulates media displacement from the media carrier. A separate air regulator, connected to the other side of the T-adaptor, controls air flow to the airbrush. This system controls the amount of media delivered to the airbrush, and is separate from the regulator controlling airflow to the airbrush. When using a larger media carrier, it is sometimes desirable to use a magnetic stirring bar and magnetic stirring plate to keep cells and other bioactive ingredients in the media in suspension.

Using an airbrush to deliver tissue engineering media allows the user to efficiently and precisely deliver layers of tissue engineering media. Previous methods of delivery were crude and gave inconsistent results. Media delivery parameters such as temperature, air pressure and nozzle diameter are integrated and offer the operator a broad range of controls to design an optimum set of delivery conditions for each desirable application, e.g., cells, scaffold type, and every specific surgical repair procedure. The delivery system is designed in a simple and straightforward manner that allows an operator to learn to use the system quickly. And a large variety of fluid media can be delivered with the invention.

It will be understood by those skilled in the art that any tissue engineering medium may be delivered by the system and method of the invention. The term, “tissue engineering medium” is well understood in the art and includes such media as, e.g., any eukaryotic cell or progenitor mammalian cell culture in a suitable carrier medium [e.g., hepatocyte, pancreatic Islet cell, fibroblast, chondrocyte, osteoblast, exocrine cell, cell of intestinal origin, bile duct cell, parathyroid cell, thyroid cell, cells of the adrenal-hypothalamic-pituitary axis, heart muscle cell, kidney epithelial cell, kidney tubular cell, kidney basement membrane cell, nerve cell, blood vessel cell, cell forming bone and cartilage, smooth muscle cell, skeletal muscle cell, ocular cell, integumentary cell, keratinocyte, transgenic cell or stem cell], growth factor, nutrient cell growth media, nutrient cell culture media, nutrient tissue culture media, nutrient tissue growth media, polymeric material such as, e.g., polycarbonate, polyarylate, block copolymer of a polycarbonates with a poly (alkylene oxide), block copolymer of polyarylate with poly (alkylene oxide), poly-alpha-hydroxycarboxylic acid, poly (capro-lactone), poly (hydroxybutyrate), polyanhydride, poly (ortho ester), polyester, and bisphenol-A based poly (phosphoester), natural or synthetic polymeric material meant to prevent tissue growth, natural or synthetic polymeric material meant to induce or enhance tissue growth, natural or synthetic polymeric material meant to wet a tissue surface, natural or synthetic polymeric material meant to be conducive to tissue growth or meant to replace, repair or enhance tissue form or function, within a mammalian system. Hydrogel systems such as alginate, gelatin and hyaluronic and hyaluronate salts may also be delivered by the system of the invention.

The following prior art outlines the history of Tissue Engineering and documents the acceptance of the term as a term of art:

Nyman S, Bone regeneration using the principles of guided tissue regeneration.

J Clin Periodontol 1991; 18:494-498.

Bell E, Ehrlich P, Buttle D J, Nakatsuji T.

Living tissue formed in vitro and accepted as skin-equivalent of full-thickness.

Science 221, 1052-1054.

Boyce S T, Glatter R, Kitsmiller J.

Treatment of chronic wounds with cultured skin substitutes: A pilot study.

Wounds: Compend. Clin. Res. Pract. 7, 24-29.

Eaglstein W H, Falanga V.

Tissue engineering and the development of Apligraf, a human skin equivalent.

Clin. Ther. 19, 894-905.

Eaglstein W H, Iriondo M, Laszlo K.

A composite skin substitute (Graftskin) for surgical wounds: A clinical experience.

Dermatol. Surg. 21, 839-843.

Hefton J M, Madden M R, Finkelstein J L, Shires G T.

Grafting of burn patients with allografts of cultured epidermal cells.

Lancet 2, 428-430.

Zacchi V, Soranzo C, Cortivo R, Radice M, Brun P, Abatangelo G.

In vitro engineering of human skin-like tissue.

J Biomed. Mater. Res. 40, 187-194.

Robert P, Frank R.

Periodontal guided tissue regeneration with a new resorbable polylactic acid membrane, J Periodontol. 65, 414-422.

Kodama T, Minabe M, Hori T.

The effect of concentration of collagen barrier on periodontal wound healing.

J Periodontol. 60, 205-210.

Martin G, Bene M C, Mole N et al.

Synthetic extracellular matrix supports healing of muco-gingival donor sites.

Tissue Eng. 1, 279-288.

Nyman S, Gottlow J, Lindhe J.

New attachment formation by guided tissue regeneration.

J Periodontol. Res. 22, 252-254.Caffesse R G, Smith B A, Castelli W A.

New attachment achieved by guided tissue regeneration in beagle dogs.

J Periodotnol. 59, 589-594.

Boyce S T, Warden G D.

Principles and practices for treatment of cutaneous wounds with cultured skin substitutes. Am J Surg. April 2002; 183(4):445-56.

Badiavas E V, Paquette D, Carson P, Falanga V.

Human chronic wounds treated with bioengineered skin: histologic evidence of host-graft interactions. J Am Acad Dermatol. April 2002; 46(4):524-30.

Lee W P. What's new in plastic surgery. J Am Coll Surg. March 2002; 194(3):324-34.

Stanton R A, Billmire D A.

Skin resurfacing for the burned patient.

Clin Plast Surg. January 2002; 29(1):29-51.

Peacock M E, Cuenin M F, Mott D A, Hokett S D.

Treatment of gingival recession with collagen membranes.

Gen Dent. January-February 2001; 49(1):94-7.

Kim T S, Holle R, Hausmann E, Eickholz P.

Long-term results of guided tissue regeneration therapy with non-resorbable and bioabsorbable barriers. II. A case series of infrabony defects.

J Periodontol. April 2002; 73(4):450-9.

Cebotari S, Walles T, Sorrentino S, Haverich A, Mertsching H.

Guided tissue regeneration of vascular grafts in the peritoneal cavity.

Circ Res. May 3, 2002; 90(8):e71.

Donos N, Kostopoulos L, Karring T.

Augmentation of the mandible with GTR and onlay cortical bone grafting.

Clin Oral Implants Res. April 2002; 13(2):175-184.

Matsumoto T, Okazaki M, Inoue M, Ode S, Chang-Chien C, Nakao H, Hamada Y, Takahashi J., Biodegradation of carbonate apatite/collagen composite membrane and its controlled release of carbonate apatite.

J Biomed Mater Res. Jun. 15, 2002;60(4):651-6.

Atala A., New methods of bladder augmentation.

BJU Int. May 2000; 85 Suppl 3:24-34; discussion 36.

Iwata H, Sakano S, Itoh T, Bauer T W.

Demineralized bone matrix and native bone morphogenetic protein in orthopaedic surgery. Clin Orthop. February 2002;(395):99-109.

Badiavas E V, Paquette D, Carson P, Falanga V.

Human chronic wounds treated with bioengineered skin: histologic evidence of host-graft interactions, J Am Acad Dermatol. April 2002; 46(4):524-30.

Rose F R, Oreffo R O, Bone tissue engineering: hope vs hype.

Biochem Biophys Res Commun. Mar. 22, 2002; 292(1):1-7.

Lee C J, Moon K D, Choi H, Woo J I, Min B H, Lee K B.

Tissue engineered tracheal prosthesis with acceleratedly cultured homologous chondrocytes as an alternative of tracheal reconstruction.

J Cardiovasc Surg (Torino). April 2002; 43(2):275-9.

Vacanti J P, Clinical implications of tissue engineering.

Harv Dent Bull. 1998 Summer; 7(2):20-2.

Hadlock T A, Vacanti J P, Cheney M L.

Tissue engineering in facial plastic and reconstructive surgery.

Facial Plast Surg. 1998; 14(3):197-203.

The media may be delivered in vivo to any desired site in the mammalian body, e.g., soft or hard tissue or in vitro to virtually any substrate such as, e.g., dental tissue, muscle tissue, dermal and/or epidermal tissue (integument), mucosal tissues, and osseous tissues. The tissues may be physiologically normal or could be damaged, wounded or traumatized.

Thus, cells can be cultured in a traditional manner and loaded into the media carrier. The tissue engineering media delivery system and method of the invention may be used to microencapsulate cells, seed cells onto scaffolds for in vitro tissue engineering research, and align collagen matrices for biomineralization studies.

Delivery via airbrush differs from delivery via pipette in that delivery via pipette produces a stream of fluid using air or another fluid (usually a gas) to displace and/or propel the desired media; and airbrush delivery combines air or other fluid (usually a gas) with the media to produce an aerosolized or partially aerosolized flow of media.

The airbrush system of the invention comprises at least a desired media container, propellant fluid, propellant-carrying hoses or other channel to connect the propellant with the delivery device, and a delivery device. Propellant fluid is generally air, but can be any customized fluid mixture, a liquid or any gas compound or mixture with the purpose of combining with the desired media, fully or partially aerosolizing or atomizing the media, and delivering the media. The delivery device can be hand held or mounted, and may mix internally or externally the desired media with air or another fluid (propellant) for the purpose of delivering the media-propellant mixture in a controlled manner.

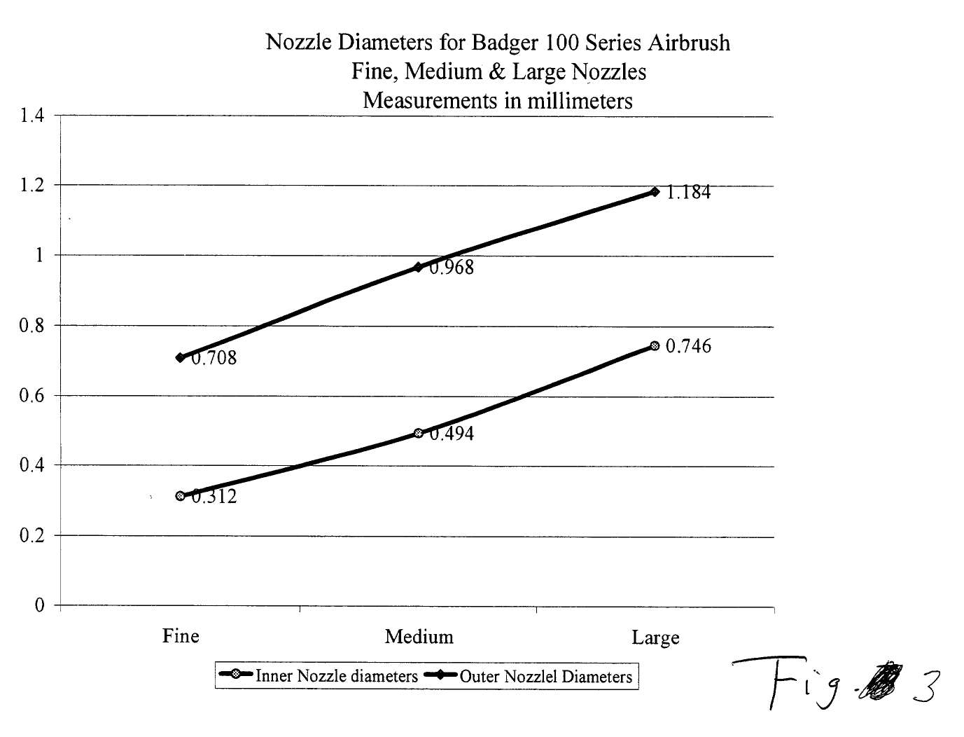

The airbrush component of the system of the invention may comprise any conventional such device such as, e.g., those marketed under the tradename Badger®. FIG. 3 sets forth nozzle diameters (±5%) for Badger* 100 series airbrushes.

### EXAMPLE 1

Bovine dermal fibroblasts were delivered into a 6-well tissue culture plate using a Badger 100 G airbrush having fine, medium and large nozzles at air pressures 6, 8, 10, 14 and 18 psi. Cells were delivered in a concentration of 200,000 cells per 1 mL of media; 1 mL of this cell suspension was delivered for each repetition. The cells were washed in Hanks Balanced Salt Solution (HBSS) or Phosphate Buffered Saline (PBS). Trypsin (0.25%) was used to detach the cells from the cell culture plate before delivery. Cells were re-suspended in a 50%-50% Dulbecco's Modified Eagle's Medium (DMEM) and F-12 media (commercially available as DMEM/F12) with 10% Fetal Bovine Serum (FBS), 2 mM L-Glutamine and 0.1 mM 2-Me.

The cells may be delivered in a variety of media, and one skilled in the art will recognize that HBSS or PBS could be used alone, or one could use DMEM/F12 alone, or any other type of vital media to deliver cells.

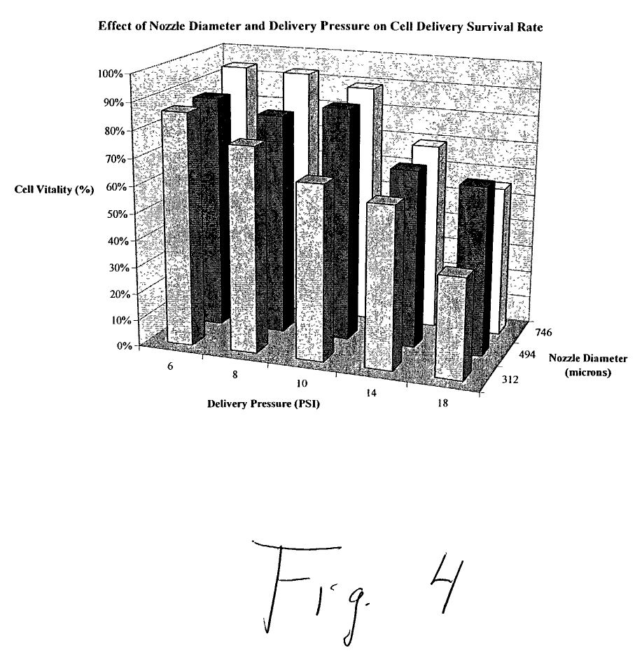

Cell vitality was measured using a trypan blue exclusion assay. The thickness of deposition was not measured. The 1 mL of airbrush delivers media spread in the tissue culture well as media would normally do. The results of cell viability after delivery are set forth in Table 1 and depicted in FIG. 4.

TABLE 1
Average Delivery Values for Cell Delivery Study using Tissue
Engineering Media Delivery System

| | Fine Nozzle | Medium Nozzle | Large Nozzle |
|---|---|---|---|
| | 312 microns | 494 microns | 746 microns |
| 6 PSI | 86% | 87% | 94% |
| 8 PSI | 76% | 82% | 93% |
| 10 PSI | 65% | 86% | 89% |
| 14 PSI | 60% | 66% | 69% |
| 18 PSI | 37% | 62% | 56% |

### EXAMPLE 2

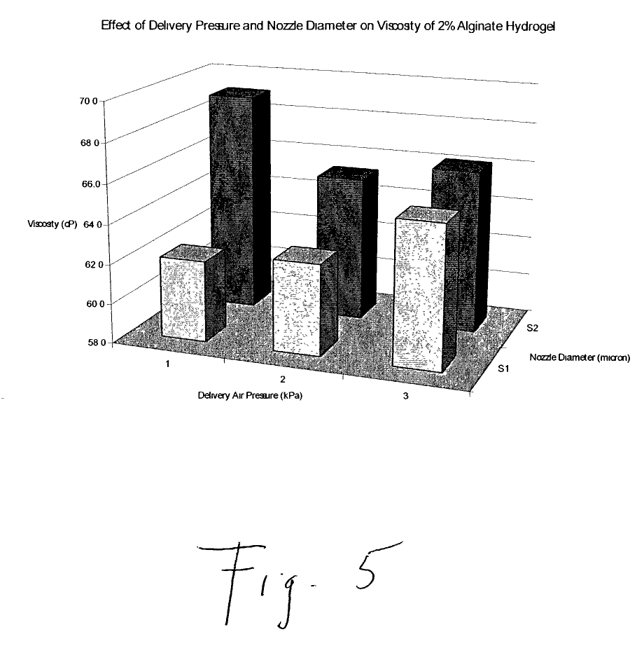

A 2% alginate hydrogel was prepared, and 2 mL of this gel was delivered using a Badger 100 G airbrush having large and medium nozzles with 10, 14 and 18 PSI delivery air pressure. A Brookfield CAP 2000 cone-and-plate rotoviscometer was used to measure the viscosity of the delivered alginate gels. Results from a portion of the large-scale hydrogel delivery study are depicted in FIG. 5.

[image]

Raw Data from Completed Cell Delivery Study #1:

| | Inner | | | | |
|---|---|---|---|---|---|
| | Nozzle | PSI | | | |
| | Diameter | (Delivery | | | % |
| Repitition | (Microns) | Pressure) | Viable | NonViable | Viable |
| 1 | 312 | 6 | 97 | 19 | 84% |
| 2 | 312 | 6 | 58 | 12 | 83% |
| 3 | 312 | 6 | 63 | 13 | 83% |
| 4 | 312 | 6 | 55 | 12 | 82% |
| 5 | 312 | 6 | 60 | 6 | 91% |
| 6 | 312 | 6 | 52 | 4 | 93% |
| 7 | 312 | 6 | 52 | 9 | 85% |
| 8 | 312 | 6 | 50 | 9 | 85% |
| 9 | 312 | 6 | 61 | 6 | 91% |
| 1 | 312 | 8 | 64 | 12 | 84% |
| 2 | 312 | 8 | 36 | 9 | 80% |
| 3 | 312 | 8 | 61 | 17 | 78% |
| 4 | 312 | 8 | 46 | 28 | 62% |
| 5 | 312 | 8 | 72 | 24 | 75% |
| 6 | 312 | 8 | 49 | 25 | 66% |
| 7 | 312 | 8 | 67 | 16 | 81% |
| 8 | 312 | 8 | 79 | 17 | 82% |
| 9 | 312 | 8 | 89 | 28 | 76% |
| 1 | 312 | 10 | 89 | 34 | 72% |
| 2 | 312 | 10 | 84 | 32 | 72% |
| 3 | 312 | 10 | 52 | 26 | 67% |
| 4 | 312 | 10 | 70 | 44 | 61% |
| 5 | 312 | 10 | 58 | 27 | 68% |
| 6 | 312 | 10 | 52 | 27 | 66% |
| 7 | 312 | 10 | 43 | 42 | 51% |
| 8 | 312 | 10 | 44 | 24 | 65% |
| 9 | 312 | 10 | 38 | 23 | 62% |
| 1 | 312 | 14 | 81 | 44 | 65% |
| 2 | 312 | 14 | 59 | 46 | 56% |
| 3 | 312 | 14 | 21 | 26 | 45% |
| 4 | 312 | 14 | 55 | 38 | 59% |
| 5 | 312 | 14 | 58 | 27 | 68% |
| 6 | 312 | 14 | 61 | 43 | 59% |
| 7 | 312 | 14 | 62 | 29 | 68% |
| 8 | 312 | 14 | 70 | 63 | 53% |
| 9 | 312 | 14 | 74 | 36 | 67% |
| 1 | 312 | 18 | 53 | 97 | 35% |
| 2 | 312 | 18 | 34 | 57 | 37% |
| 3 | 312 | 18 | 50 | 50 | 50% |
| 4 | 312 | 18 | 38 | 64 | 37% |
| 5 | 312 | 18 | 44 | 49 | 47% |
| 6 | 312 | 18 | 30 | 74 | 29% |
| 7 | 312 | 18 | 35 | 62 | 36% |
| 8 | 312 | 18 | 29 | 74 | 28% |
| 9 | 312 | 18 | 50 | 94 | 35% |
| 1 | 494 | 6 | 91 | 13 | 88% |
| 2 | 494 | 6 | 101 | 21 | 83% |
| 3 | 494 | 6 | 75 | 15 | 83% |
| 4 | 494 | 6 | 107 | 12 | 90% |
| 5 | 494 | 6 | 81 | 10 | 89% |
| 6 | 494 | 6 | 113 | 15 | 88% |
| 7 | 494 | 6 | 111 | 21 | 84% |
| 8 | 494 | 6 | 98 | 13 | 88% |
| 9 | 494 | 6 | 84 | 13 | 87% |
| 1 | 494 | 8 | 72 | 16 | 82% |
| 2 | 494 | 8 | 89 | 25 | 78% |
| 3 | 494 | 8 | 104 | 17 | 86% |
| 4 | 494 | 8 | 91 | 18 | 83% |
| 5 | 494 | 8 | 98 | 11 | 90% |
| 6 | 494 | 8 | 99 | 15 | 87% |
| 7 | 494 | 8 | 68 | 19 | 78% |
| 8 | 494 | 8 | 77 | 19 | 80% |
| 9 | 494 | 8 | 64 | 26 | 71% |
| 1 | 494 | 10 | 104 | 15 | 87% |
| 2 | 494 | 10 | 83 | 23 | 78% |
| 3 | 494 | 10 | 85 | 9 | 90% |
| 4 | 494 | 10 | 62 | 11 | 85% |
| 5 | 494 | 10 | 102 | 13 | 89% |
| 6 | 494 | 10 | 80 | 4 | 95% |
| 7 | 494 | 10 | 97 | 17 | 85% |
| 8 | 494 | 10 | 66 | 10 | 87% |
| 9 | 494 | 10 | 56 | 16 | 78% |
| 1 | 494 | 14 | 67 | 31 | 68% |
| 2 | 494 | 14 | 64 | 29 | 69% |
| 3 | 494 | 14 | 50 | 21 | 70% |
| 4 | 494 | 14 | 62 | 37 | 63% |
| 5 | 494 | 14 | 66 | 31 | 68% |
| 6 | 494 | 14 | 62 | 40 | 61% |
| 7 | 494 | 14 | 76 | 38 | 67% |
| 8 | 494 | 14 | 69 | 28 | 71% |
| 9 | 494 | 14 | 59 | 45 | 57% |
| 1 | 494 | 18 | 69 | 32 | 68% |
| 2 | 494 | 18 | 56 | 31 | 64% |
| 3 | 494 | 18 | 54 | 42 | 56% |
| 4 | 494 | 18 | 53 | 36 | 60% |
| 5 | 494 | 18 | 67 | 43 | 61% |
| 6 | 494 | 18 | 44 | 24 | 65% |
| 7 | 494 | 18 | 57 | 32 | 64% |
| 8 | 494 | 18 | 73 | 40 | 65% |
| 9 | 494 | 18 | 44 | 30 | 59% |
| 1 | 746 | 6 | 72 | 1 | 99% |
| 2 | 746 | 6 | 71 | 1 | 99% |
| 3 | 746 | 6 | 53 | 2 | 96% |
| 4 | 746 | 6 | 68 | 7 | 91% |
| 5 | 746 | 6 | 68 | 10 | 87% |
| 6 | 746 | 6 | 66 | 5 | 93% |
| 1 | 746 | 8 | 89 | 1 | 99% |
| 2 | 746 | 8 | 77 | 4 | 95% |
| 3 | 746 | 8 | 49 | 5 | 91% |
| 4 | 746 | 8 | 62 | 7 | 90% |
| 5 | 746 | 8 | 43 | 6 | 88% |
| 6 | 746 | 8 | 71 | 1 | 99% |
| 7 | 746 | 8 | 66 | 8 | 89% |
| 8 | 746 | 8 | 68 | 4 | 94% |
| 9 | 746 | 8 | 65 | 5 | 93% |
| 1 | 746 | 10 | 61 | 4 | 94% |
| 2 | 746 | 10 | 74 | 9 | 89% |
| 3 | 746 | 10 | 45 | 9 | 83% |
| 4 | 746 | 10 | 30 | 10 | 75% |
| 5 | 746 | 10 | 71 | 5 | 93% |
| 6 | 746 | 10 | 78 | 7 | 92% |
| 7 | 746 | 10 | 63 | 5 | 93% |
| 8 | 746 | 10 | 50 | 5 | 91% |
| 9 | 746 | 10 | 67 | 6 | 92% |
| 1 | 746 | 14 | 69 | 12 | 85% |
| 2 | 746 | 14 | 54 | 13 | 81% |
| 3 | 746 | 14 | 36 | 21 | 63% |
| 4 | 746 | 14 | 45 | 29 | 61% |
| 5 | 746 | 14 | 58 | 26 | 69% |
| 6 | 746 | 14 | 48 | 13 | 79% |
| 7 | 746 | 14 | 46 | 17 | 73% |
| 8 | 746 | 14 | 47 | 27 | 64% |
| 9 | 746 | 14 | 32 | 32 | 50% |
| 1 | 746 | 18 63 | 52 | 55% | |
| 2 | 746 | 18 | 72 | 54 | 57% |
| 3 | 746 | 18 | 49 | 49 | 50% |
| 4 | 746 | 18 | 42 | 50 | 46% |
| 5 | 746 | 18 | 82 | 68 | 55% |
| 6 | 746 | 18 | 50 | 33 | 60% |
| 7 | 746 | 18 | 63 | 40 | 61% |
| 8 | 746 | 18 | 44 | 28 | 61% |
| 9 | 746 | 18 | 70 | 53 | 57% |

Hydrogel Study I Raw Data

| | diameter | pressure | cP |
|---|---|---|---|
| replicate | (microns) | (PSI) | (centiPoisies) |
| 1 | 746 | 18 | 61.9 |
| 2 | 746 | 18 | 61.9 |
| 3 | 746 | 18 | 61.9 |
| 4 | 746 | 18 | 61.9 |
| 5 | 746 | 18 | 63.8 |
| 6 | 746 | 18 | 60 |
| 7 | 746 | 18 | 61.9 |
| 8 | 746 | 18 | 64.7 |
| 9 | 746 | 18 | 60.9 |
| 1 | 746 | 14 | 61.9 |
| 2 | 746 | 14 | 62.8 |
| 3 | 746 | 14 | 62.8 |
| 4 | 746 | 14 | 61.9 |
| 5 | 746 | 14 | 62.8 |
| 6 | 746 | 14 | 61.9 |
| 7 | 746 | 14 | 62.8 |
| 8 | 746 | 14 | 63.8 |
| 9 | 746 | 14 | 62.8 |
| 1 | 746 | 10 | 62.8 |
| 2 | 746 | 10 | 64.7 |
| 3 | 746 | 10 | 64.7 |
| 4 | 746 | 10 | 65.6 |
| 5 | 746 | 10 | 65.6 |
| 6 | 746 | 10 | 65.6 |
| 7 | 746 | 10 | 63.8 |
| 8 | 746 | 10 | 65.6 |
| 9 | 746 | 10 | 68.4 |
| 1 | 494 | 18 | 65.6 |
| 2 | 494 | 18 | 63.8 |
| 3 | 494 | 18 | 65.8 |
| 4 | 494 | 18 | 71.3 |
| 5 | 494 | 18 | 75 |
| 6 | 494 | 18 | 75 |
| 7 | 494 | 18 | 75 |
| 8 | 494 | 18 | 65.6 |
| 9 | 494 | 18 | 64.7 |
| 1 | 494 | 14 | 65.6 |
| 2 | 494 | 14 | 65.6 |
| 3 | 494 | 14 | 62.8 |
| 4 | 494 | 14 | 65.6 |
| 5 | 494 | 14 | 68.4 |
| 6 | 494 | 14 | 64.7 |
| 7 | 494 | 14 | 66.6 |
| 8 | 494 | 14 | 65.6 |
| 9 | 494 | 14 | 62.8 |
| 1 | 494 | 10 | 67.5 |
| 2 | 494 | 10 | 65.6 |
| 3 | 494 | 10 | 67.5 |
| 4 | 494 | 10 | 66.6 |
| 5 | 494 | 10 | 63.8 |
| 6 | 494 | 10 | 66.6 |
| 7 | 494 | 10 | 66.6 |
| 8 | 494 | 10 | 65.6 |
| 9 | 494 | 10 | 65.6 |

[image]

1. A method for tissue engineering comprising the steps of:
(1) providing a flowable biocompatible composition; and
(2) delivering said composition to a substrate via fluid propulsion, said flowable composition comprising a tissue engineering medium and said composition being delivered by fluid propulsion under conditions not substantially deleterious to said tissue engineering medium.

2. The method of claim 1 wherein said fluid propulsion comprises airbrush delivery.

3. The method of claim 1 wherein said tissue engineering medium is a biodegradable scaffold forming material.

4. The method of claim 3 wherein said composition is treated after delivery to form a biodegradable scaffold in vivo for regeneration of tissue.

5. The method of claim 4 wherein said composition forms, after said treatment, a biocompatible and biodegradable polymer.

6. The method of claim 5 wherein said polymer is a polycarbonate, polyarylate, block copolymer of a polycarbonates with a poly (alkylene oxide), block copolymer of polyarylate with poly (alkylene oxide), poly-alpha-hydroxycarboxylic acid, poly (capro-lactone), poly (hydroxybutyrate), polyanhydride, poly (ortho ester), polyester, and bisphenol- A based poly (phosphoester).

7. The method of claim 1 wherein said composition is delivered by a micro air brush.

8. The method of claim 1 wherein said tissue engineering medium is delivered in vitro.

9. The method of claim 1 wherein said tissue engineering medium is delivered in vivo to a body site.

10. The method of claim 9 wherein said body site is soft tissue.

11. The method of claim 9 wherein said body site is hard tissue.

12. The method of claim 1 wherein said tissue engineering medium includes at least one bioactive agent.

13. The method of claim 12 wherein at least one of said bioactive agents is a cell.

14. The method of claim 13, wherein said cell is hepatocyte, pancreatic Islet cell, fibroblast, chondrocyte, osteoblast, exocrine cell, cell of intestinal origin, bile duct cell, parathyroid cell, thyroid cell, cells of the adrenal-hypothalamic-pituitary axis, heart muscle cell, kidney epithelial cell, kidney tubular cell, kidney basement membrane cell, nerve cell, blood vessel cell, cell forming bone and cartilage, smooth muscle cell, skeletal muscle cell, ocular cell, integumentary cell, keratinocyte, transgenic cell or stem cell.

15. A system for delivering tissue engineering media to a substrate comprising container means for storing tissue engineering media, airbrush means for delivering said tissue engineering media to a desired substrate and means for transferring said tissue engineering media from said container to said airbrush.

16. The system of claim 15 wherein said transfer means is a microcentrifuge tube.

17. The system of claim 15 additionally comprising means for separately regulating air supply and tissue engineering media to said airbrush.
